(19) United States
(12) Patent Application Publication
(10) Pub. No.: US 2023/0372127 A1
(43) Pub. Date: Nov. 23, 2023

(54) Prosthetic Liner Assist Device

(71) Applicants: United States Government As Represented By The Department Of Veterans Affairs, Washington, DC (US); The Administration of the Tulane Educational Fund, New Orleans, LA (US)

(72) Inventors: William B. Layman, New Orleans, LA (US); Kali Dancisak, Kenner, LA (US); Erika Kudryk, Tampa, FL (US); Edward Moy, Franklin, TN (US); Alan Shepardson, Lakeworth, FL (US)

(21) Appl. No.: 18/303,883

(22) Filed: Apr. 20, 2023

Related U.S. Application Data
(60) Provisional application No. 63/332,719, filed on Apr. 20, 2022.

(51) Int. Cl. A61F 2/78

(52) U.S. Cl. CPC A61F 2/7812; A61F 2002/7825

(57) ABSTRACT

A device for assisting amputees donning prosthetic liners uses a plurality of elongate fingers, radially biased about a central space, to support the liner against the residual limb as the liner is applied. Rollers on each finger provide a frictionless traversal of the device by the limb as the limb is inserted into the central space. Hinged connection of the fingers to a base supporting the fingers, as well as hinges allowing each finger to bend, ensure that engagement between the rollers and the liner is maintained during both limb insertion and withdrawal. Elastic bungees or shock cords joining the fingers provide the radial biasing to the fingers toward the central space.

### CROSS-REFERENCE TO RELATED APPLICATION

This application claims priority to and the benefit of the filing date of U.S. Provisional Patent Application No. 63/332,719, filed Apr. 20, 2022, the contents of which are incorporated herein by reference in their entirety.

### FIELD

The application is generally related to prosthetic devices and, in particular, to devices and methods of use hereof to assist amputees in donning prosthetic liners for use with artificial limbs.

### BACKGROUND

Amputees who have difficulty donning prosthetic liners need an affordable, versatile, and accessible way to accurately and comfortably don their residual limb liners. Donning a standard prosthetic liner traditionally requires the use of both hands, grip strength in both hands, and the ability to bend over for lower extremity amputees. For older amputees who have decreased mobility or amputees that are also missing fingers or hands, this presents a problem. There is clearly a need for a hands-free way for amputees to don their residual limb liners.

### SUMMARY

Embodiments of the present device offer assistance to amputees donning lower body prosthetic liners. For example, some embodiments provide a prosthetic liner holder device having a plurality of elongate resilient fingers extending upwardly from a base. The plurality of elongate fingers are configured to retain a prosthetic liner thereon in an orientation that allows the amputee to don the prosthetic liner by inserting the corresponding residual limb into the liner and arrangement of resilient elongate fingers on which it is disposed. Upon withdrawal of the residual limb from the arrangement of resilient fingers, the prosthetic liner is transferred onto the user's residual limb in a proper alignment and fit.

Additionally, alternate embodiments of the device may include a stand on which the liner holder is mounted so that a seated amputee may position the liner holder in a desired position without having to maintain a grip on the liner holder during insertion of the residual limb. The stand allows for adjustment of both the height and angle of the liner holder prior to the amputee donning the liner. As such, an amputee in a wheelchair or like device may don the prosthetic liner without the need to bend over during the operation.

Additional advantages of the invention will be set forth in part in the description that follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION OF THE DRAWINGS

These and other features of the preferred embodiments of the invention will become more apparent in the detailed description in which reference is made to the appended drawings wherein:

FIGS. 1 and 2 are isometric views of an example device for assisting donning prosthetic liners according to the disclosure;

FIGS. 3, 4, and 5 are isometric views of example components used in the device shown in FIGS. 1 and 2;

FIGS. 6-11 are isometric views illustrating a simulated use of the device shown in FIGS. 1 and 2;

FIG. 12 is an isometric view of another example embodiment of a device for assisting donning prosthetic liners according to the disclosure;

FIGS. 13 and 14 are plan views of a portion of a component used with an example device according to the disclosure;

FIG. 15 is an isometric view of a component used with an example device according to the disclosure;

FIG. 16 is an isometric view of a portion of an example device embodiment;

FIGS. 17A and 17B are isometric views of the device shown in FIGS. 1 and 2 including elongate rigid handles;

FIGS. 18-20 are isometric views of an example stand for use with the device shown in FIGS. 1 and 2;

FIG. 21 is an isometric view of an example angle bracket of the stand shown in FIGS. 18-20;

FIG. 22 is a partial perspective view of the device shown in FIGS. 1 and 2;

FIGS. 23-25 are isometric views of the device shown in FIGS. 1 and 2 installed on the stand shown in FIGS. 18-20; and

FIGS. 26A-26C are isometric and schematic views of an alternate example device for assisting with donning prosthetic liners;

FIG. 27 is an isometric view of an alternate example device for assisting with donning prosthetic liners; and

FIG. 28 is an isometric view of an alternate example device for assisting with donning prosthetic liners.

### DETAILED DESCRIPTION

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout. It is to be understood that this invention is not limited to the particular methodology and protocols described, as such may vary. It is also to be understood that the terminology used herein is for the purpose of describing particular embodiments only, and is not intended to limit the scope of the present invention.

Many modifications and other embodiments of the invention set forth herein will come to mind to one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

As used herein the singular forms “a,” “an,” and “the” include plural referents unless the context clearly dictates otherwise. For example, use of the term “a finger” can refer to one or more of such fingers, and so forth.

All technical and scientific terms used herein have the same meaning as commonly understood to one of ordinary skill in the art to which this invention belongs unless clearly indicated otherwise.

As used herein, the terms “optional” or “optionally” mean that the subsequently described event or circumstance may or may not occur, and that the description includes instances where said event or circumstance occurs and instances where it does not.

As used herein, the term “at least one of” is intended to be synonymous with “one or more of” For example, “at least one of A, B and C” explicitly includes only A, only B, only C, and combinations of each.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Optionally, in some aspects, when values are approximated by use of the antecedents “about,” “substantially,” or “generally,” it is contemplated that values within up to 15%, up to 10%, up to 5%, or up to 1% (above or below) of the particularly stated value can be included within the scope of those aspects. In other aspects, when angular values are approximated by use of the antecedents “about,” “substantially,” or “generally,” it is contemplated that angular values within up to 15 degrees, up to 10 degrees, up to 5 degrees, or up to one degree (above or below) of the particularly stated angular value can be included within the scope of those aspects.

The word “or” as used herein means any one member of a particular list and, unless context dictates otherwise, can also include any combination of members of that list.

In the following description and claims, wherever the word “comprise” or “include” is used, it is understood that the words “comprise” and “include” can optionally be replaced with the words “consists essentially of” or “consists of” to form another embodiment.

It is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

The following description supplies specific details in order to provide a thorough understanding. Nevertheless, the skilled artisan would understand that the apparatus, system, and associated methods of using the apparatus can be implemented and used without employing these specific details. Indeed, the apparatus, system, and associated methods can be placed into practice by modifying the illustrated apparatus, system, and associated methods and can be used in conjunction with any other apparatus and techniques conventionally used in the industry.

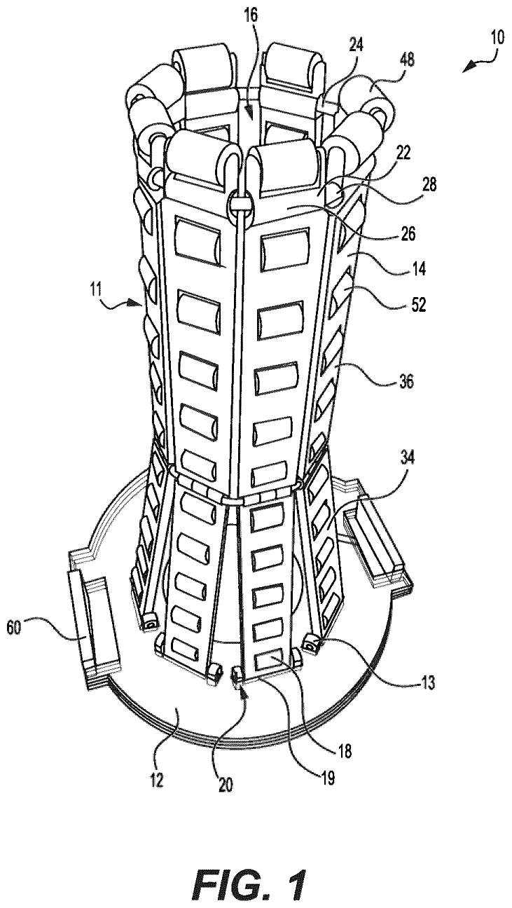

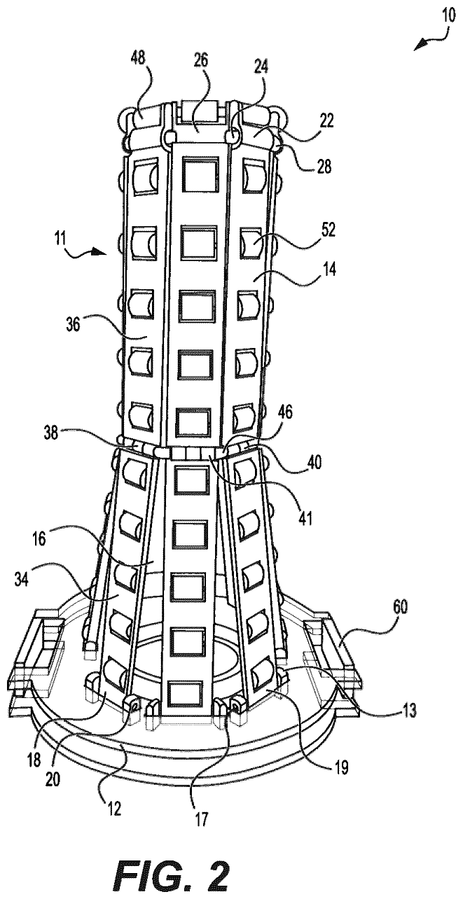

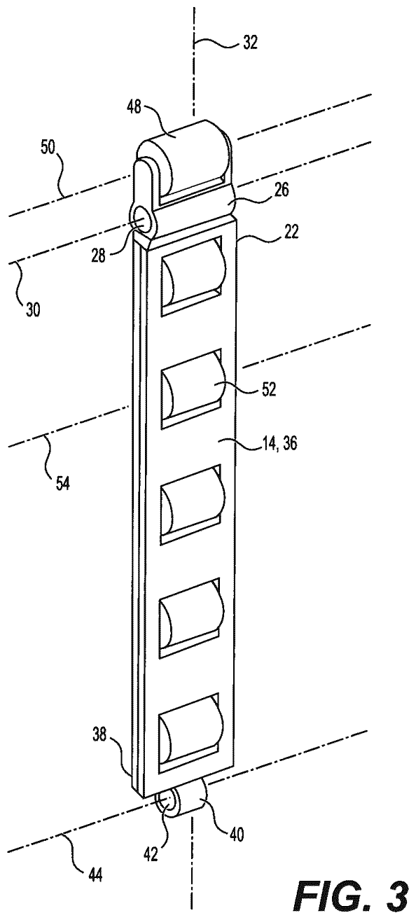

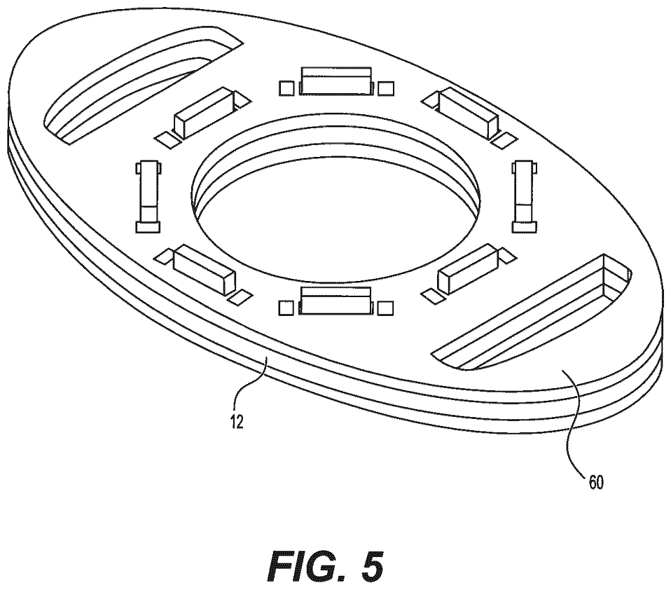

The present disclosure is directed to a device to assist an amputee in donning a prosthetic liner. As shown in FIGS. 1 and 2, an example embodiment of a device 10 according to the disclosure comprises a liner holder 11 having a base 12 (see also FIG. 5) and a plurality of elongate fingers 14 arranged concentrically about a central space 16. In this example, each finger 14 comprises a lower end 18 pivotably attached to the base 12. Pivotable attachment of fingers 14 to base 12 is achieved by respective hinges 20 acting between each finger 14 and the base 12. As shown, each hinge 20 includes a barrel 19 secured between a pair of arms 13 by a pin 17 extending therethrough. Each finger 14 further comprises an upper end 22. Each upper end of each finger is elastically connected to two adjacent fingers thereby permitting the fingers 14 to move radially outwardly and inwardly away from and toward the central space 16. The elastic connection between the fingers 14 is effected by an elastic member 24 which is constrained by respective barrels 26 positioned proximate the upper end 22 of each finger. As shown in FIG. 3, each barrel 26 defines a respective bore 28 there through. Each bore 28 is concentric with a respective axis 30 oriented transversely to a longitudinal axis 32 of each finger. As shown in FIGS. 1 and 2, the elastic member 24 extends through each bore 28 to elastically connect the upper ends 22 of the fingers 14 to one another. In this example embodiment, the elastic member 24 is substantially continuous, meaning that its ends are joined to one another after the elastic member is passed through the bores 28. In a practical example, the elastic member 24 comprises an elastic core surrounded by a braided sheath, commercially known as a bungee or shock cord.

As shown in FIG. 2, each finger 14 of the example embodiment comprises a lower portion 34, which includes the finger's lower end 18, and an upper portion 36, which includes the finger's upper end 22. A hinge 38 attaches the upper portion 36 to the lower portion 34. Hinges 38 are respectively positioned between the lower and upper ends 18 and 22 of fingers 14, in this example, approximately at a mid-point of each finger. Each hinge 38 is oriented to permit motion of each finger radially toward and away from the central space 16.

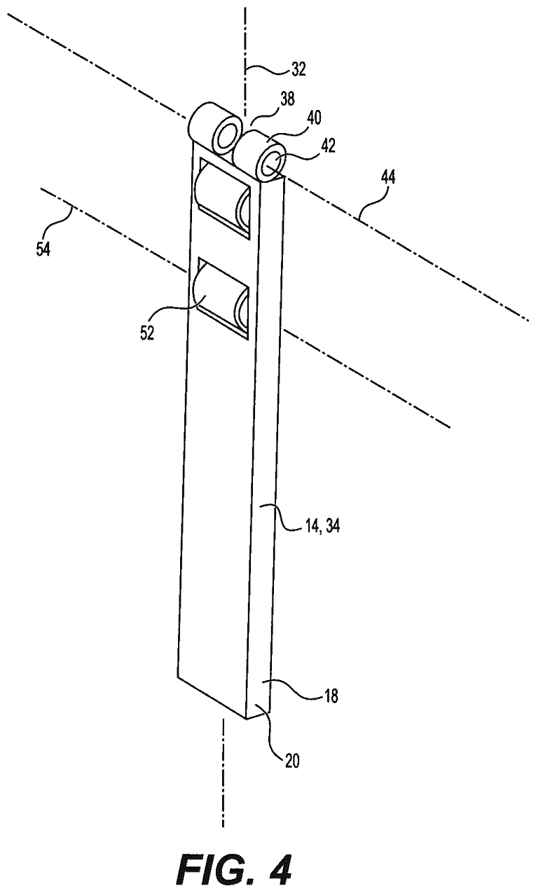

Fingers 14 are further elastically connected to one another between the upper and lower ends 22 and 18. To effect this elastic connection, as shown in FIGS. 3 and 4, each finger 14 comprises a respective barrel 40 positioned proximate to the hinge 38. Each barrel 40 defines a respective bore 42 there through. Each bore 42 is concentric with a respective axis 44 oriented transversely to the longitudinal axis 32 of each finger 14. As shown in FIG. 2, an elastic member 46 extends through each bore 42. The elastic member 46 elastically connects the fingers to one another between the upper and lower ends 22 and 18. Again, the elastic member 46 is substantially continuous and in a practical example comprises an elastic core surrounded by a braided sheath. Efficiency and simplicity are achieved in the example embodiment when each barrel 40 is integrally formed with a respective one of the hinges 38. As shown in FIGS. 2, 3 and 4, the respective bore axes 44 of barrels 40 align as the barrels engage one another, allowing the elastic member 46 to act as a hinge pin.

As shown in FIGS. 1 and 3, each finger 14 further comprises a roller 48 positioned proximate the finger's upper end 22. As shown in FIG. 3, each roller 48 has an axis of rotation 50 oriented transversely to the finger's longitudinal axis 32. In this example embodiment, each roller 48 has a cylindrical shape, although other shapes, such as spherical or conical, are also practical. It is thought further advantageous to position additional rollers 52 in spaced relation lengthwise along each finger 14. As shown in FIG. 3, each roller 52 also has an axis of rotation 54 oriented transversely to the longitudinal axis 32 of each finger 14. Again, in a practical embodiment, each roller 52 may have a cylindrical shape, other shapes as noted above also being practical.

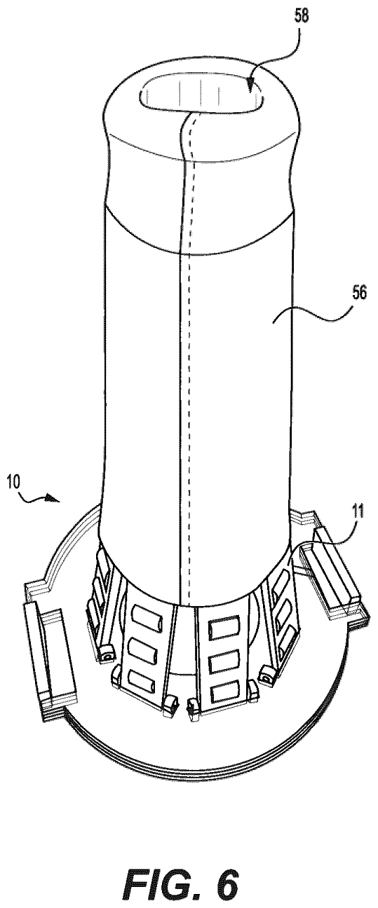

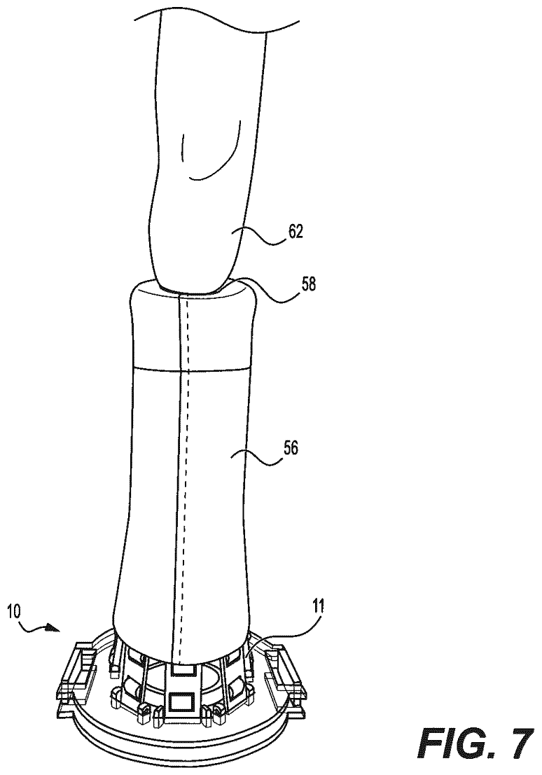

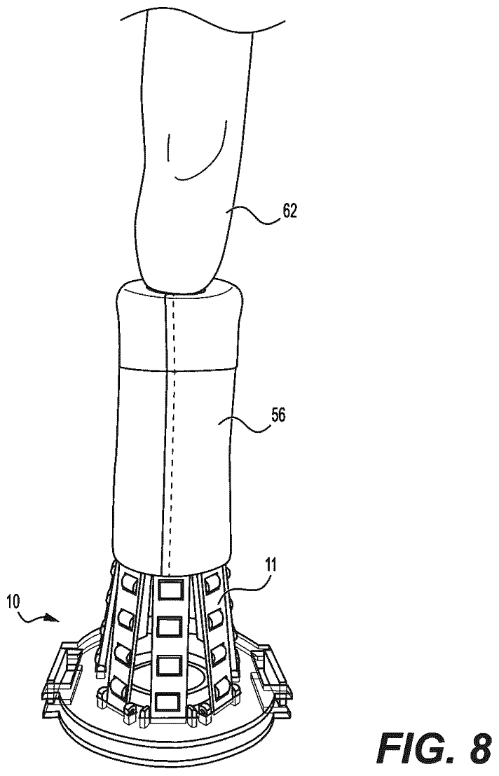

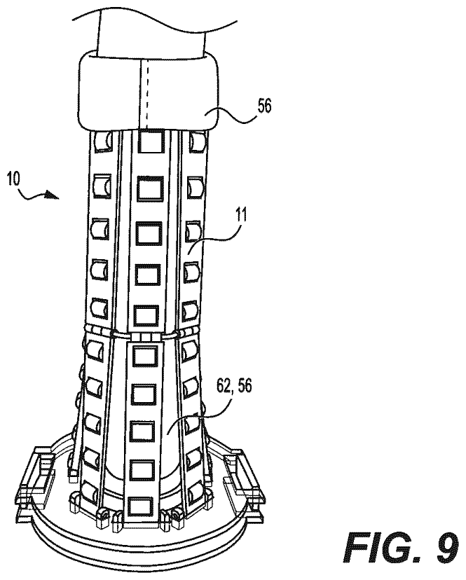

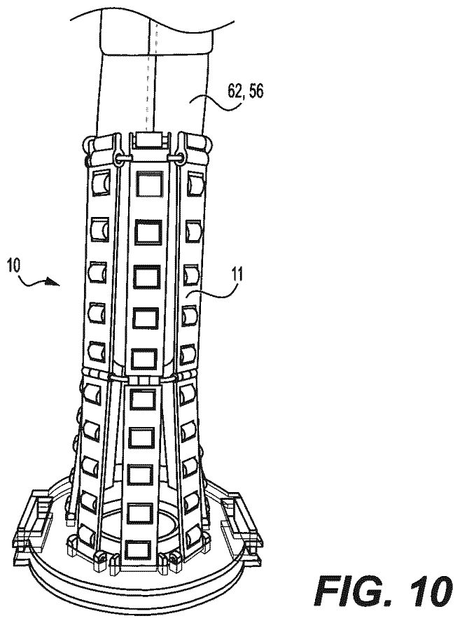

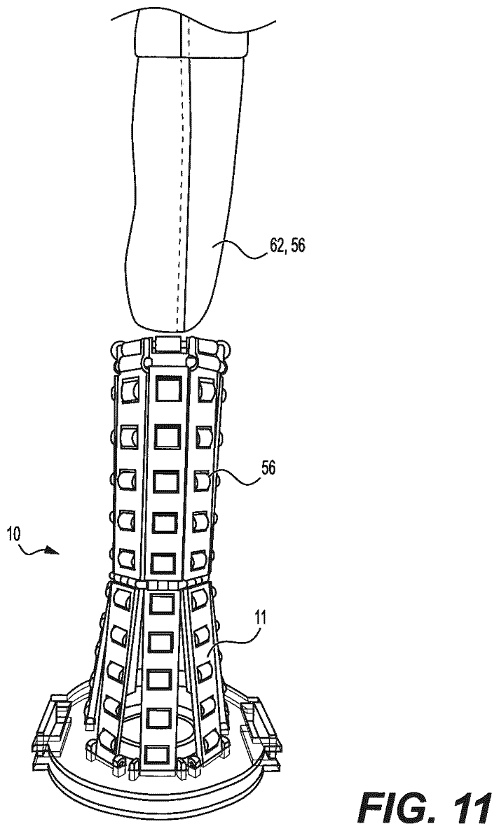

FIGS. 1 and 6-11 illustrate a simulated use of the example device 10. As shown in FIG. 6, the prosthetic liner 56 is placed inside out on the device 10. Positioning of the liner 56 is made easy using one or more colored alignment indicators (not shown) on the device 10 to ensure a proper fit. At this stage, a depression 58 may also be formed in the liner 56 by pushing a portion of the liner into the central space 16. Depression 58 acts as a centering lead-in for guiding the amputee's residual limb. As shown in FIG. 7, the amputee then pushes their residual limb 62 into the depression 58 atop device 10. As they push their limb 62 further into the device 10 (FIG. 8), the rollers 48, positioned proximate the upper end 22 of fingers 14, and the rollers 52, positioned in spaced relation lengthwise along the fingers 14, are radially biased toward the central space 16 by the elastic members 24 and 46 (see FIG. 1 for references to the device 10). The rollers 48 and 52 are thus biased into engagement with the liner 56 which is gradually and tightly applied to the residual limb 62 as it traverses device 10. As shown by a comparison of FIGS. 7 and 8, as liner 56 is forced into the central space 16, the portion of the liner 56 on the outside of device 10 moves upwardly toward the upper ends 22 of fingers 14. Rolling action of the rollers ensures a friction-free and comfortable application of the liner 56 to the residual limb 62 as it traverses the device. Once the residual limb 62 is fully engaged with the prosthetic liner 56 (FIG. 9) it may then be withdrawn (FIG. 10). During withdrawal, the rollers 48 and 52 continue to engage and support the liner 56 and permit frictionless withdrawal of the limb 62, with the liner remaining on the limb (FIG. 11).

An amputee can use the device independently by placing it on a floor or on a flat surface and stepping into it. As well, as discussed below, the device 10 may include a stand 80 that allows an amputee to use the device while seated, as in a wheelchair. Device 10 may also comprise handles 60 positioned on the base 12 (see FIGS. 1, 2 and 5), allowing it to be used by a physical therapist, a healthcare provider, prosthetist, caretaker, etc., to assist the amputee in donning their liner.

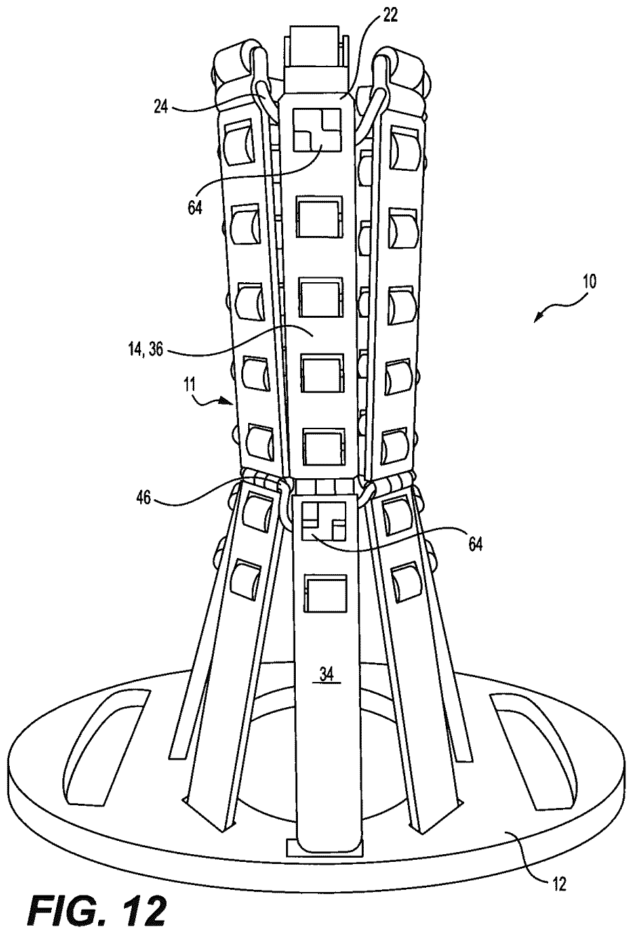

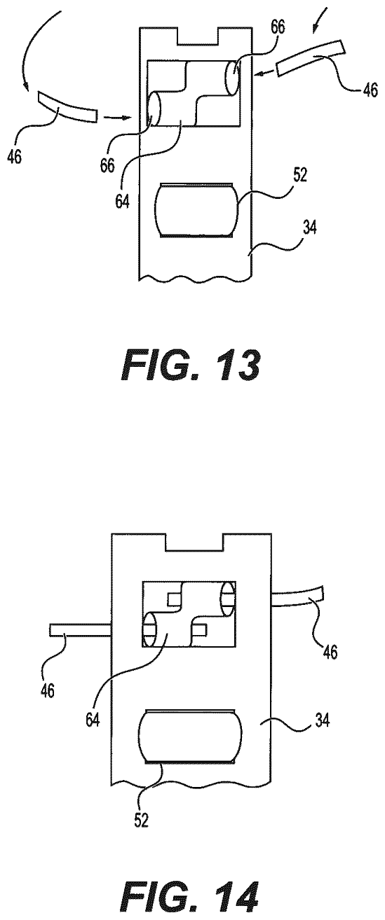

FIG. 12 shows another example embodiment of device 10 according to the disclosure wherein an upper portion 36 of a finger 14 is rendered in a different color from the other fingers to designate the “front” of the device. This feature allows the amputee to accurately align the front of device 10 with the anterior of a residual limb, such as a leg, for proper use of the device. FIG. 12 also illustrates one example for affixing the ends of the elastic members 24 and 46 to the fingers 14. Receptacles 64 are placed proximate to the upper end 22 of one finger 14 as well as the upper end of the lower portion 34 of the finger. As shown in FIGS. 13 and 14, the receptacles 64 define openings 66 which receive the free ends of the elastic members 24 and 46 (46 shown). The free ends of the elastic members are held in place within receptacles 64 using an adhesive, such as cyanoacrylate in this example. Other means for attachment are also feasible. It is advantageous to form receptacles 64 as a cylindrical body which simulates a roller 52 and have the receptacle openings 66 facing the central space so that the device 10 presents a uniform outward appearance. FIG. 15 shows another example finger embodiment 14 wherein the receptacle 64 is positioned directly adjacent to the roller 48 on the upper portion 36 so that the line of the elastic member 24 is better maintained as it encircles the device 10. A similar embodiment may also be used for the elastic member 46 connecting the joints between the upper and lower finger portions 36 and 34. FIG. 16 shows another way for affixing elastic member 46, using a knot 68 to join its free ends. This embodiment is advantageous because it permits ready adjustment of pre-tension in the elastic member, as well as ease of replacement of a worn or severed elastic member.

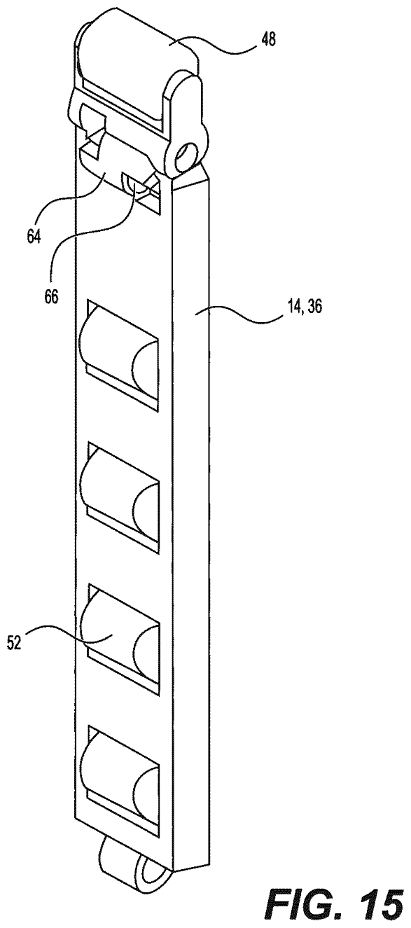

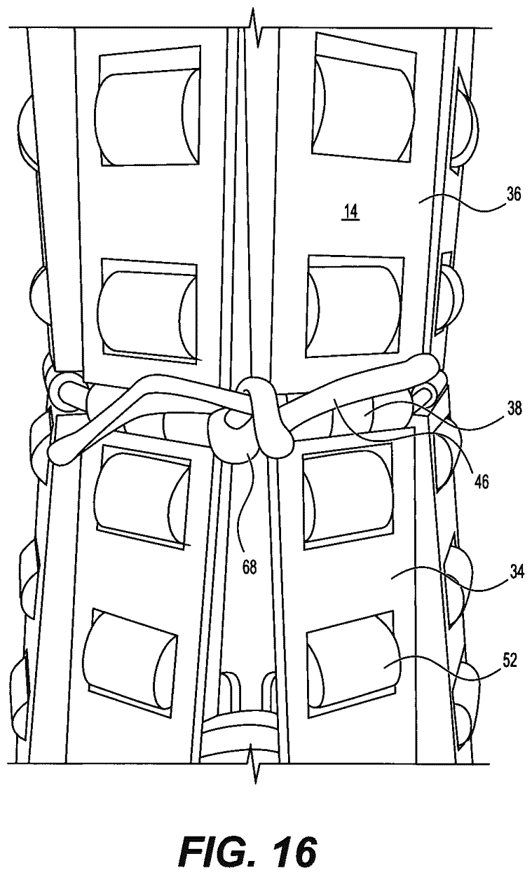

FIGS. 17A and 17B show another example embodiment of the device 10 according to the disclosure wherein a pair of elongate handles 70 are provided to assist the amputee when donning a prosthetic liner. As shown, each handle 70 includes a molded grip 72 disposed at the top of the handle and a threaded portion 74 disposed at the bottom end of the handle. The threaded portion 74 of each handle 70 is configured to be removably received in a correspondingly threaded bore 76 formed in the base 12 of the device 10 on opposites of the formation of elongate fingers 14. The elongate handles 70 allow the user to lift and manipulate the device 10 without having to bend over.

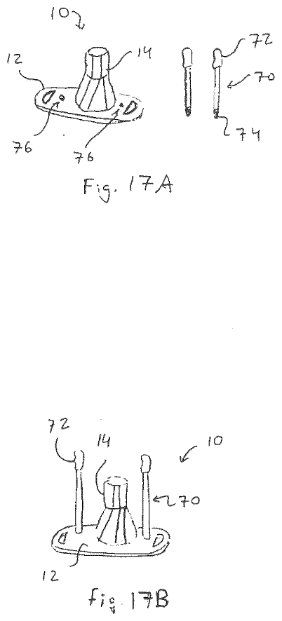

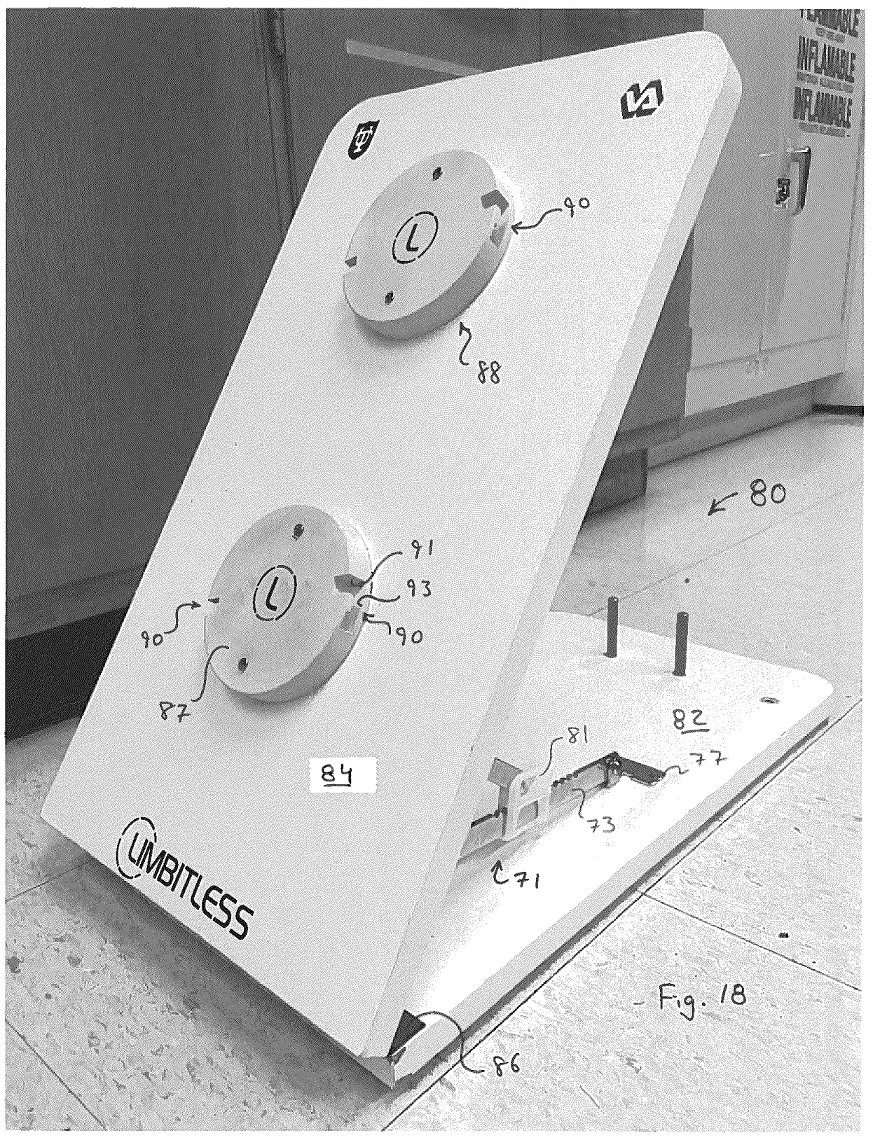

Referring now to FIGS. 18 through 20, embodiments of the device according to the disclosure may also include a stand 80 to act as an adjustable foundation for the liner holder 11. As shown, the stand 80 assists in positioning the liner holder 11 in such a way that a seated amputee may be able to utilize the liner holder 11 to don a liner 56 (FIG. 6) without assistance. The adjustability of the stand 80 allows for a variety of heights and angles to be chosen so that the amputee can personalize the overall donning device 10 to fit their preferences.

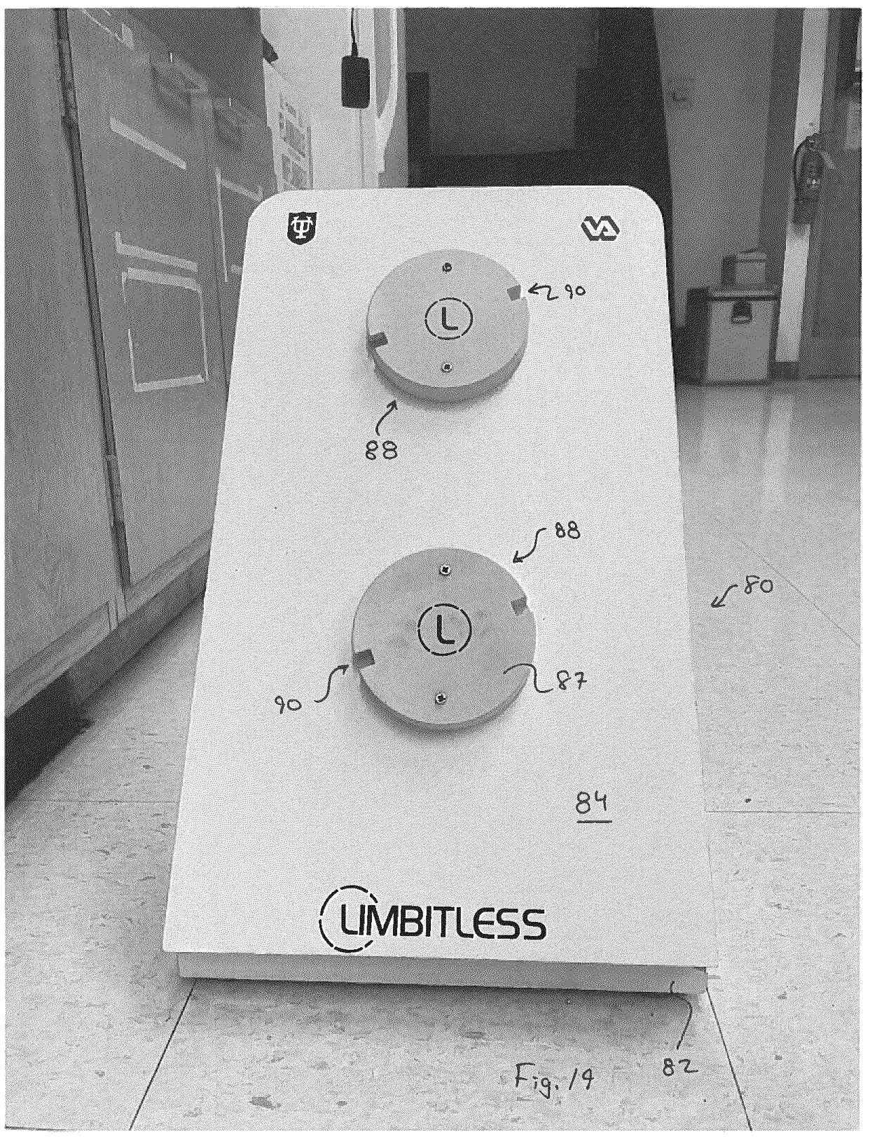

The stand 80 includes a base platform 82 and an adjustable top platform 84 that are connected to each other at adjacent elongate edges by a hinge 86 so that the top platform 84 is pivotable with respect to the base platform 82. As best seen in FIG. 21, an angle bracket 71 secures the base platform 82 to the top platform 84. The angle bracket 71 allows the user to secure the top platform 84 up to an angle of 50° from the base platform 82. Preferably, one or more liner mounts 88 extend outwardly from the upper surface of the top platform 84 and act as mounting points for the liner holder 11.

Referring specifically to FIG. 21, the angle bracket 71 preferably includes a bottom arm 73 and a top arm 75 that are pivotably secured at their distal ends to the base platform 82 and the top platform 84, respectively, by mounting brackets 77, as shown in FIG. 20. An angle arm 79 is pivotably secured to the top arm 75 at a first end and to a lock block 81 at a second end. The lock block 81 is slidably adjustable along a toothed rack 83 disposed along the top edge of the bottom arm 73, the number of teeth 85 and spacing therebetween along the rack 83 determining the range of degrees and degree interval between positions of the top platform 84 with respect to the base platform 82. To adjust the angle of the top platform 84 with respect to the base platform 82, the user lifts up on the top platform 84 until the lock bar 87 of the lock block 81 is disengaged from the rack 83. While maintaining slight upward force, the user then urges the top platform 84 either away or toward themself to either decrease or increase, respectively, the angle formed between the upper platform 84 and the base platform 82. When the desired position is reached, the user lets go of the top platform 84 so that the lock bar 87 re-engages one of the gaps 93 formed between the teeth 85 of the rack 83.

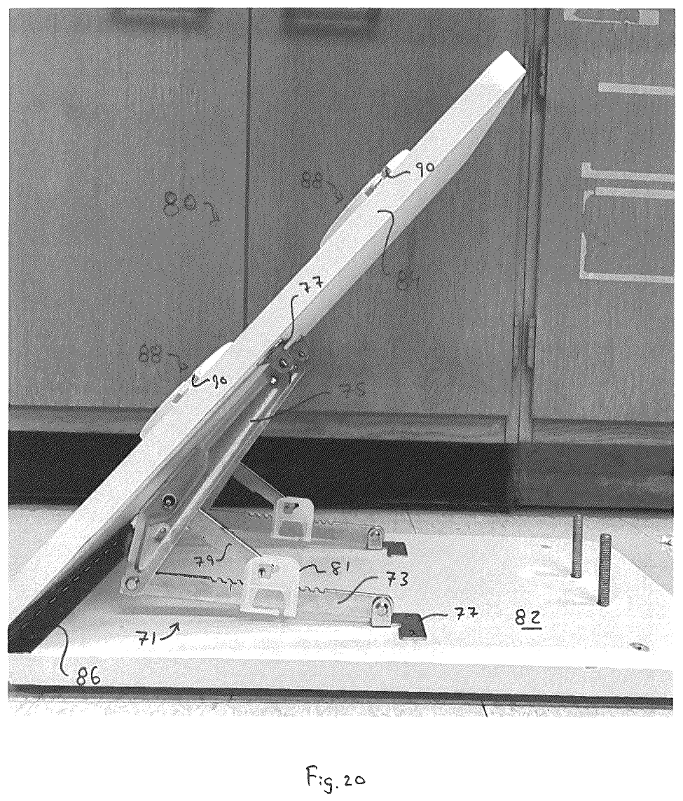

As shown, each liner mount 88 is substantially cylindrical in shape and includes a bayonet-style mounting arrangement which permits secure connection of the liner holder 11 to the stand 80. Each bayonet connector includes at least two slots 90 formed in the cylindrical sidewall 89 of each liner mount 88, each slot 90 including a vertical portion 91 extending inwardly from the face 87 of the liner mount 88 to a circumferential portion 93 that extends along the side wall 89. Each slot is configured to slidably receive a corresponding tab 92 (FIG. 22) that extends radially-inwardly from the perimeter of an aperture 94 formed in the base 12 of the liner holder 11. The aperture 94 is configured to receive a liner mount therein.

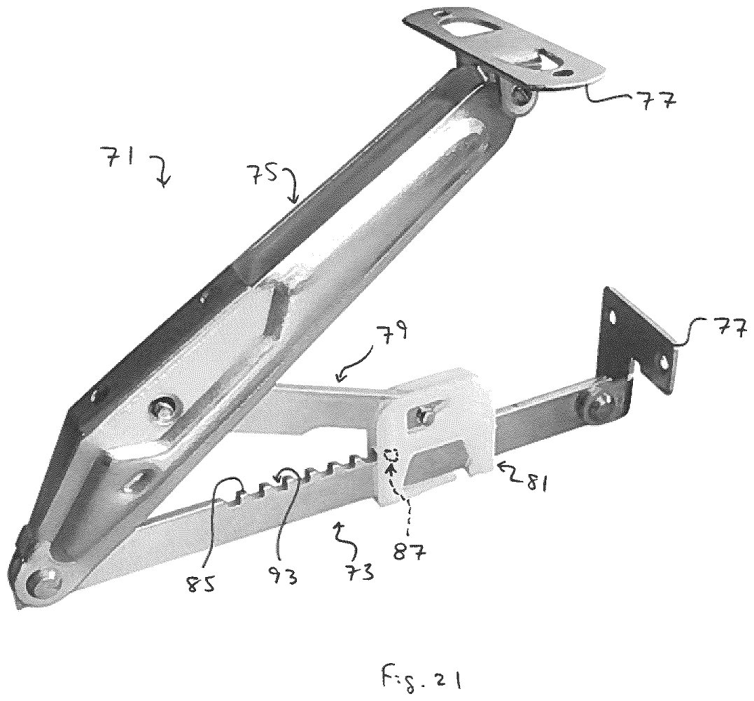

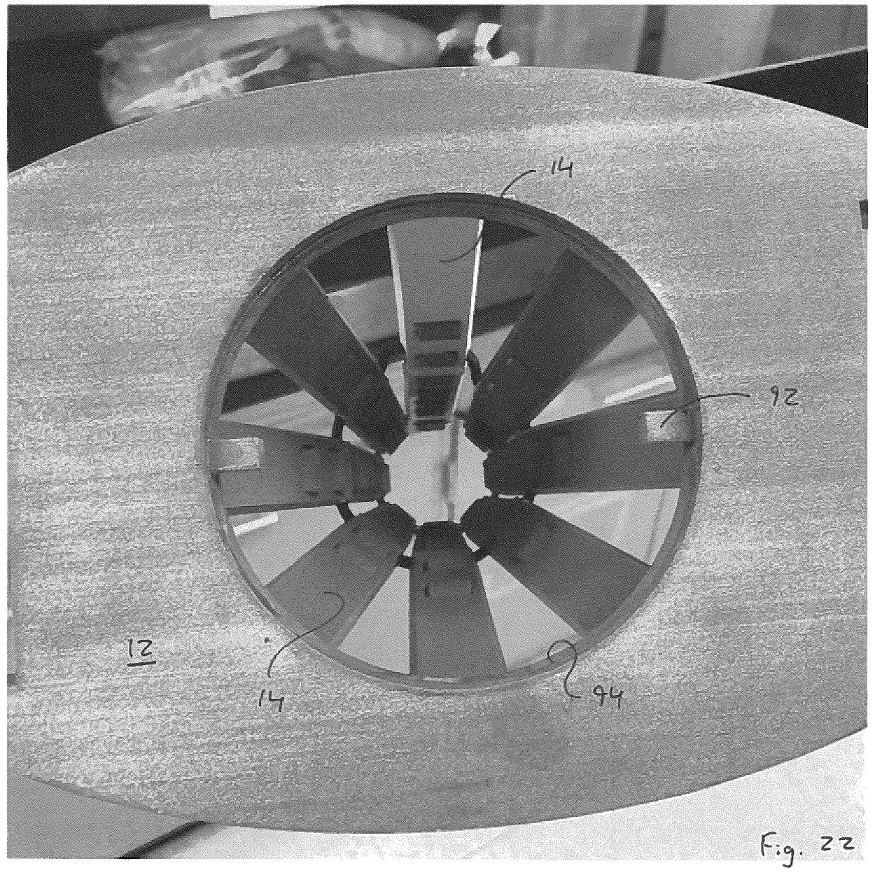

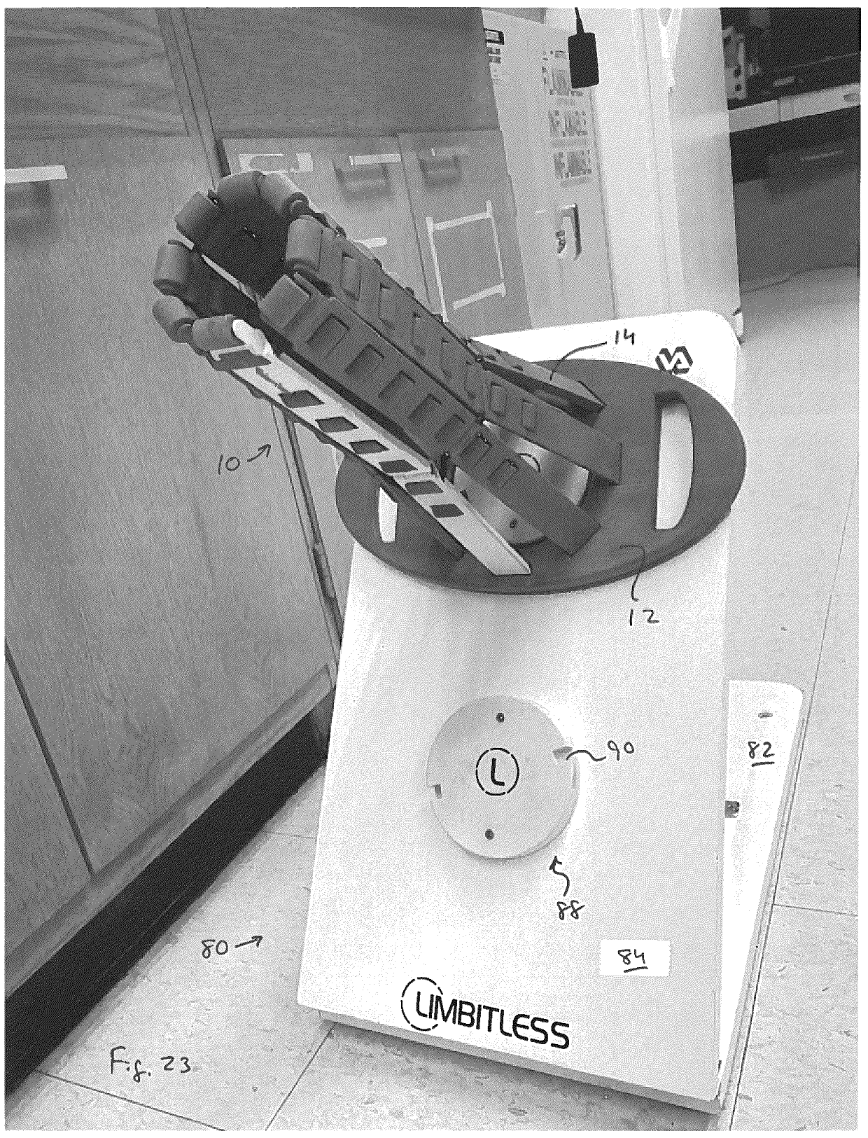

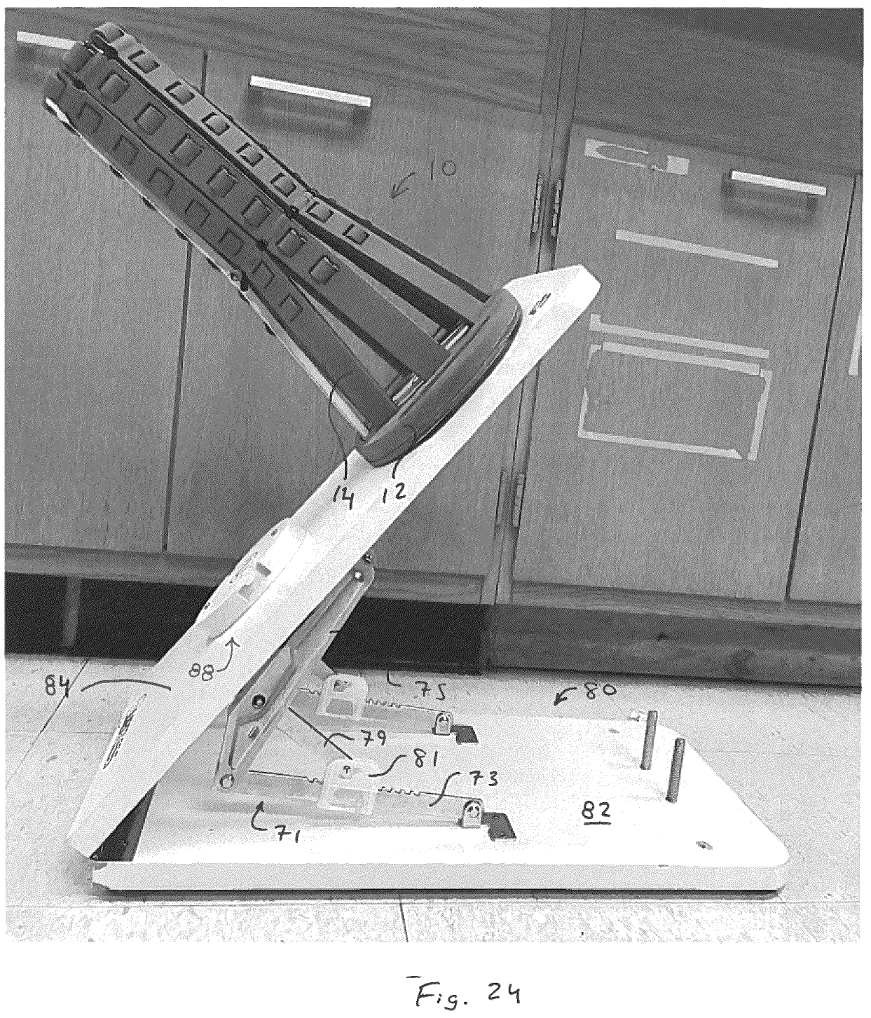

Referring additionally to FIGS. 23 through 25, to secure the liner holder 11 to a liner mount 88, the tabs 92 are aligned with and inserted into the vertical portions 91 of the slots 90 formed in the corresponding liner mount 88. Next, the base 12 is rotated in the clockwise direction so that the tabs 92 slide along the circumferential portions 93 of the slots 90. Engagement of the tabs 92 with the slots 90 allows the amputee to withdraw the residual limb from the liner holder 11 without the liner holder 11 pulling away from the stand 80. Additionally, when used, the elongate handles 70 (FIGS. 17A and 17B) allow the amputee to exert downward force on liner holder 11 while withdrawing the corresponding limb to prevent movement of the stand 80. The process for donning a prosthetic liner 56 using the stand 80 is substantially the same as the process discussed above with regard to FIGS. 6-11. To remove the liner holder 11, the user merely reverses the noted steps for mounting.

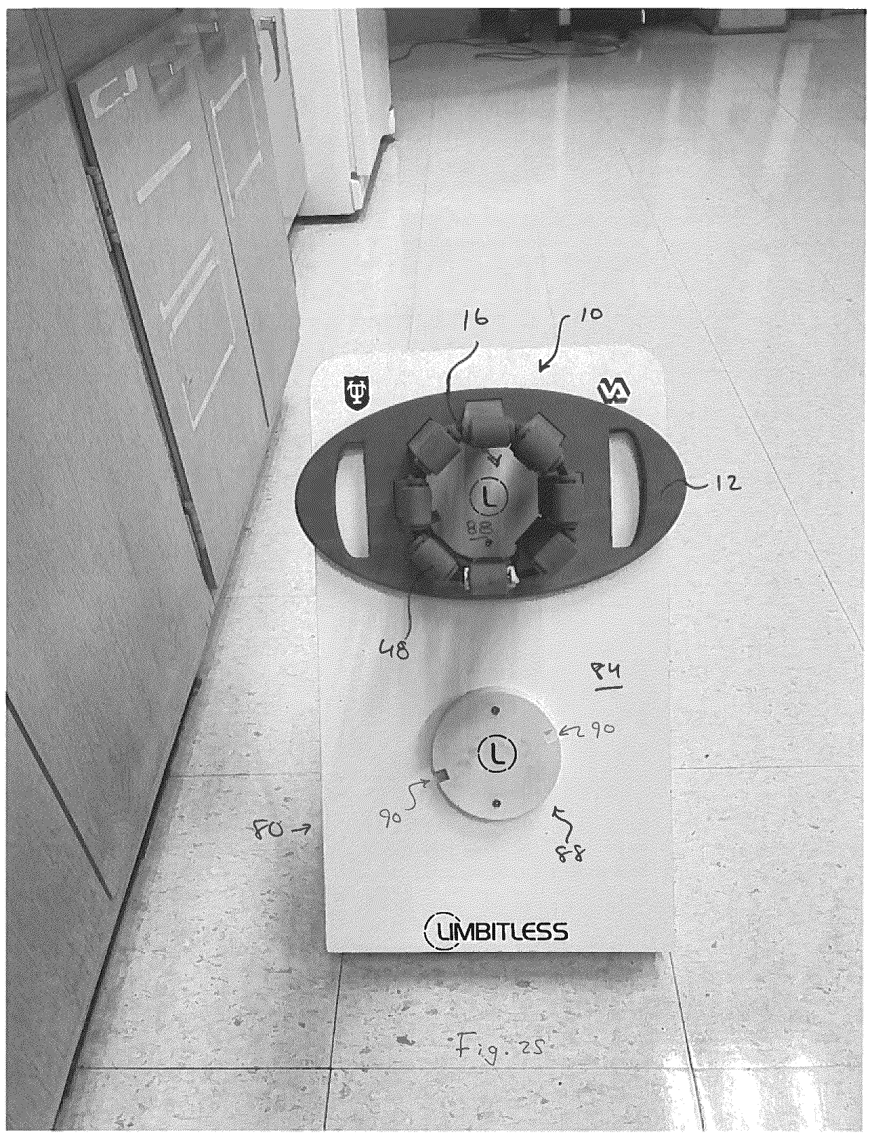

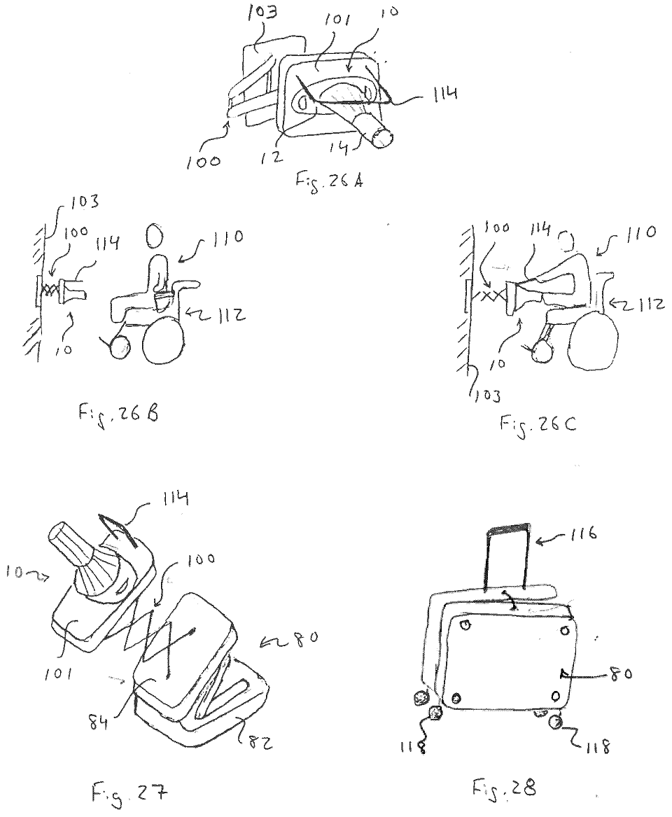

Referring now to FIGS. 26A-26C, in yet another embodiment of the disclosed device 10 for donning a prosthetic liner, the base 12 of the liner holder is secured to a platform 101 that is secured to a wall 103 by an articulated arm 100. As best seen in FIGS. 26B and 26C, the device 10 is positioned at a height that is aligned with the residual limb of the amputee 110. As shown, the amputee 110 is seated in a wheelchair 112 and is able to don a prosthetic liner by aligning his residual limb with the device 10 and pulling the device toward the residual limb by utilizing a handle 114 on the device 10. After the residual limb is fully inserted in the device 10 and the prosthetic liner donned, the amputee pushes the device away using the handle 91.

In yet other embodiments of the device 10, the articulated arm 100 may be used to secure the liner holder and platform 101 to the top panel 84 of the previously discussed stand 80 (FIG. 18). This configuration further prevents the necessity of bending over to properly don the prosthetic liner. As shown in FIG. 28, in various other embodiments of the device 10, a carry handle 116 and wheels 118 may be provided to assist with transporting the stand 80 in its collapsed configuration.

Compared to traditional devices and methods for prosthetic liner application, example devices 10 according to the disclosure are expected to provide a more accurate fit on the first try, to reduce potential injury relating to liner misfit (such as chafing or suction bruises) as well as decrease the time required to don the liner. It is also expected that the use of device 10 to easily and comfortably apply a prosthetic liner will encourage amputees to wear their prostheses on a more regular basis, thereby increasing their physical activity and positively affecting their health. It is envisioned that devices 10 will be made available alone or in kit form to include at least one prosthetic liner.

Although the foregoing invention has been described in some detail by way of illustration and example for purposes of clarity of understanding, certain changes and modifications may be practiced within the scope of the appended claims.

What is claimed is:

1. A device to assist donning of a prosthetic liner, said device comprising:
a holder device configured to removeably receive the prosthetic liner, comprising:
a base;
a plurality of elongate fingers arranged concentrically about a central space, each said finger comprising:
a lower end pivotably attached to said base; and
an upper end;
wherein each said upper end of each said finger is elastically connected to two adjacent said fingers thereby permitting said fingers to move radially outwardly and inwardly away from and toward said central space.

2. The device according to claim 1, wherein each said finger comprises a hinge acting between said finger and said base.

3. The device according to claim 1, wherein each said finger comprises:
a lower portion which includes said lower end;
an upper portion which includes said upper end;
a hinge attaching said upper portion to said lower portion, said hinge being positioned between said lower and upper ends and oriented to permit motion of each said finger radially toward and away from said central space.

4. The device according to claim 3, wherein said hinge is positioned at a mid-point of each said finger.

5. The device according to claim 1, wherein each said finger comprises a roller positioned proximate said upper end, each said roller having an axis of rotation oriented transversely to a longitudinal axis of said finger.

6. The device according to claim 5, wherein each said roller has a cylindrical shape.

7. The device according to claim 1, wherein each said finger comprises a plurality of rollers, said rollers being positioned in spaced relation lengthwise along said finger, each said roller having an axis of rotation oriented transversely to a longitudinal axis of said finger.

8. The device according to claim 7, wherein each said roller has a cylindrical shape.

9. The device according to claim 1, wherein each said finger comprises a respective barrel positioned proximate said upper end, each said barrel defining a respective bore there through, each said bore being concentric with a respective axis oriented transversely to a longitudinal axis of each said finger; wherein
an elastic member extends through each said bore, said elastic member elastically connecting said upper ends of said fingers to one another.

10. The device according to claim 9, wherein said elastic member is substantially continuous.

11. The device according to claim 10, wherein said elastic member comprises an elastic core surrounded by a braided sheath.

12. The device according to claim 3, wherein each said finger comprises a respective barrel positioned proximate said hinge, each said barrel defining a respective bore there through, each said bore being concentric with a respective axis oriented transversely to a longitudinal axis of each said finger; wherein
an elastic member extends through each said bore, said elastic member elastically connecting said fingers to one another between said upper and lower ends thereof.

13. The device according to claim 12, wherein said elastic member is substantially continuous.

14. The device according to claim 13, wherein said elastic member comprises an elastic core surrounded by a braided sheath.

15. The device according to claim 12, wherein each said barrel is integrally formed with a respective one of said hinges.

16. The device according to claim 1, further comprising:
a stand comprising:
a base platform;
a top platform that is pivotably secured to the base platform; and
a linear mount disposed on a top surface of the top platform that is configured to removably secure the holder device to the top platform,
wherein an angle defined between the top platform and the base platform is adjustable.

17. The device according to claim 16, wherein the stand further comprises:
an angle bracket comprising:
a bottom arm having a distal end pivotably secured to a top surface of the base platform; and
a top arm having a distal end pivotably secured to a bottom surface of the top platform;
wherein a proximal end of the bottom arm and a proximal end of the top arm are pivotably connected so that an angle between the top platform and the base platform are adjustable.

18. The device according to claim 17, wherein the angle bracket further comprises:
an angle arm having a first end and a second end, the first end being pivotably secured to the top arm;
a lock block including a lock bar, the lock block being pivotably secured to the second end of the angle arm;
a rack formed by a plurality of teeth disposed on a top edge of the bottom arm,
wherein the lock block is slidable along the bottom arm, and lock bar is selectively engageable with a selected one of gaps defined between adjacent teeth of the rack.

19. The device according to claim 16, wherein:
the stand further comprises a substantially cylindrical liner mount extending outwardly from the top surface of the top panel; and
the base of the holder device defines a mounting aperture that is configured to slidably receive the liner mount therein.

20. The device according to claim 19, wherein the liner mount further comprises at least one mounting slot, the mounting aperture includes at least one mounting tab extending radially-inwardly therefrom, and the at least one mounting tab is slidably receivable in the at least one mounting slot.

21. The device according to claim 19, further comprising a pair of elongate handles that extend outwardly from a top surface of the base of the holder device on opposing sides of the mounting aperture.

22. A kit comprising the device according to claim 1, the kit further comprising at least one of the prosthetic liners.
